(19) United States
(12) Patent Application Publication
(10) Pub. No.: US 2003/0171424 A1
(43) Pub. Date: Sep. 11, 2003

(54) Intravenous formulation of artelinic acid for treatment of severe and complicated malaria

(76) Inventors: Ai J. Lin, Gaithersburg, MD (US); John VanHamont, Fort Meade, MD (US); Wilbur K. Milhous, Germantown, MD (US)

Correspondence Address: Caroline Nash, Nash & Titus, LLC, 3415 Brookeville Road, Brookeville, MD, 20833, US

(21) Appl. No.: 10/376,420

(22) Filed: Feb. 27, 2003

Related U.S. Application Data
(60) Provisional application No. 60/362,986, filed on Mar. 7, 2002.

(51) Int. Cl. A61K 31/335

(52) U.S. Cl. 514/452

(57) ABSTRACT

Chemical entities of artelinic acid-L-lysine salts and their intravenous formulation for treatment of severe and complicated malaria, especially the cerebral malaria, which is fatal if not treated promptly, and method of making an intravenous artelinic acid-L-lysine salt formulation.

### PRIORITY

This application claims priority of provisional application No. 60/362,986 filed Mar. 7, 2002.

### GOVERNMENT INTEREST

The invention described herein may be manufactured, used and licensed by or for the U.S. Government.

### BACKGROUND OF THE INVENTION

1. Field of the Invention

This invention covers the chemical entities of artelinic acid-L-lysine salts (AL/LY) and its intravenous formulation, for treatment of severe and complicated malaria, especially the cerebral malaria that is fatal if not treated promptly.

2. Brief Description of Related Art

Falciparum malaria remains a major cause of human morbidity and mortality causing 300-500 million cases and an estimated 1-1.5 million deaths annually. The high mortality is attributable to severe anemia and organ dysfunction from sequestration of infected erythrocytes to post-capillary venules, causing vessel occlusion and resulting in tissue or organ ischemia. Prompt therapy with rapid acting parenteral antimalarials is part of the key to reducing morbidity and mortality. Few drugs are available intravenously for treatment of severe complicated malaria or for patients who cannot tolerate oral medications.

Malaria continues to present a major threat to U.S. military forces deployed to tropical areas of the world. Nearly all U.S. soldiers lack acquired immunity to malaria, leaving them at special risk for acquiring severe forms of malaria.

An invention is needed to treat malaria since quinine is an old remedy and the only parenteral antimalarial drug available in U.S.A today. Due to drug resistance and toxicity, and difficulty of administration, intravenous quinine has only limited efficacy in reducing the mortality rate of cerebral malaria. Furthermore, there are only a very small number of known antimalarials with sufficient efficacy, water solubility and stability that can be developed into a parenteral drug.

The effectiveness of artemisinin and its derivatives as antimalarial drugs for the treatment of multidrug resistant Plasmodium falciparum has received increasing attention in recent years.¹⁻⁶ Due to the increasing prevalence of multidrug resistant P. falciparum, several artemisinin derivatives are being used in areas such as Southeast Asia.^{4-6b} The practical values of these novel malarial therapeutic agents, nevertheless, are impaired by their (a) poor solubility in either oil or water^{3a}, (b) high rate of parasite recrudescence after treatment,^{3b} (c) short plasma half-life,^{7,8} and/or (d) poor oral activity (active only at very high dosage).^{3b}

Artemisinin derivatives differ from quinine/quinidine, the traditional treatments of severe malaria, in that they kill parasites much more rapidly. Most mortality from severe malaria occurs in the first 24-48 hours. Theoretically, artemisinin derivatives should reduce the mortality rate of cerebral malaria patients. However, several large well-controlled comparisons of artemether (AM) and arteether (AE) versus quinine showed no clear difference in morbidity or mortality from severe malaria.⁹⁻¹³ The lack of improvement may be due to the delayed peak concentration of these oil soluble derivatives by intramuscular injection. Artesunate suppositories are being developed by Mepha and the World Health Organization (WHO) for initiation of treatment of critically ill patients where medical facilities are not available, as delay in treatment is an important contributor to mortality. However, bioavailability and variability of absorption is also an issue with the suppository route of administration.^{14, 15}

Chemical modifications of artemisinin have resulted in a number of analogs with improved efficacy and increased solubility in either oil, i.e., arteether and artemether^{3a}, or water, i.e., sodium artelinate¹⁶ and sodium artesunate.¹⁷ Both artemether and arteether are more potent than artemisinin, but have short plasma half-life and produce fatal CNS toxicity in chronically dosed rats and dogs.^{18, 19} Likewise, the usefulness of sodium artesunate in the treatment of cerebral malaria and multidrug resistant P. falciparum is offset by problems associated with its instability in aqueous solution¹, the high rate of recrudescence, and the extremely short plasma half-life (20 to 30 minutes).^{18a}

The following are structures of artemisinin derivatives. Except artelinic acid (AL), all other three compounds are in clinical use today. However, artemether, arteether and artesunic acid all have problems associated with them of either toxicity or poor stability.

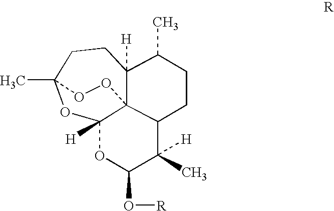

Artemether —CH₃ (Oil Soluble)

Arteether —CH₂CH₃ (Oil Soluble)

Sodium artelinate

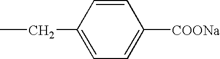

(Water Soluble)

Sodium artesunate —COCH₂CH₂COONa (Water Soluble)

Another IV injectable artemisinin derivative, AS (artesunic acid), is unstable in basic solution and is prone to hydrolyze rapidly back to water insoluble dihydroartemisinin and succinic acid. It dissolves slowly in 5% sodium bicarbonate solution with evolution of carbon dioxide to form sodium artesunate aqueous solution. During the process of dissolution, shaking and sometimes heating are necessary to accelerate the making of a clear solution. Caking, gas forming and precipitation upon standing are common phenomena which have frustrated the physicians and nurses in its clinical application. Due to its instability, the unused sodium artesunate solution decomposed quickly to form water insoluble dihydroartemisinin which precipitates out from the solution within 2-3 hours, a detrimental application problem especially in a tropical region without air conditioner. Further, there have been other toxicity problems associated with artesunate. As stated above, artesunate tends to hydrolyze back to dihydroartemisinin in vivo which was shown to penetrate into cerebrospinal fluid after administration of intravenous artesunate in severe falciparum malaria patients and the concentration of dihydroartemisinin, which is toxic, in cerebrospinal fluid increased with time.²⁰

Sodium artelinate (Na-AL) was examined in an attempt to overcome the lability problem of sodium artesunate (Na-AS) in aqueous solution. Na-AL is not only very stable in aqueous solution¹ but also has much longer plasma half-life (1.5 to 3 hrs) than sodium artesunate or oil soluble analogs.¹⁸ It is highly effective when administered orally against P. berghei²¹ and produced complete cures against P. knowlesi infection in rhesus monkeys at 15 mg/kg×3 days²². Furthermore, recent CNS toxicity studies indicated that the water soluble dihydroartemisinin derivatives such as AL and AS possess substantially less CNS toxicity in rats and dogs than oil soluble analogs such as artemether and arteether.¹⁹

Artelinic acid (AL) was patented in 1988 (Lin, et al., U.S. Pat. No. 4,791,135)² as antimalarial agents by Walter Reed Army Institute of Research and is currently under development as parenteral drug for treatment of severe and complicated malaria. Unexpectedly, AL formulation has encountered numerous problems which are unique to artelinic acid and not commonly observed with other acidic drugs.

### PREPARATION OF SODIUM SALTS

Artelinic acid is not water soluble until it was converted to a salt form. The most commonly used water soluble salt for acidic drugs is sodium salt. The sodium salt of artelinic acid was prepared in our laboratory by dissolving artelinic acid in a slightly less than one equivalent mole of sodium hydroxide solution. The slight excess amount of insoluble artelinic acid was removed by extraction of the mixture with diethyl ether. The aqueous layer was evaporated under reduced pressure using a rotary evaporator to remove the small amount of volatile diethyl ether, and then lyophilized to give sodium artelinate as a white powder. The freshly prepared sodium artelinate (Na-AL) is highly water soluble (>60 mg/ml). However, portions of the sodium artelinate were converted to free artelinic acid on exposure to atmospheric carbon dioxide and moisture which formed carbonic acid. The carbonic acid is acidic enough to convert some of the Na-AL salt back to AL free acid. Therefore, the old lot of sodium artelinate does not dissolve completely in distilled water and the insoluble material was identified by NMR as free artelinic acid.

Similar problems were observed with freshly prepared sodium artelinate aqueous solution which turned turbidity on standing at room temperature because of the formation of free AL on standing.

With the problems of Na-AL being sensitive to carbonic acid identified, early efforts on AL intravenous formulation have focussed on dissolving the Na-AL in diluted sodium carbonate solution (0.02 to 0.5%). The Na-AL is very soluble in various concentrations of the sodium carbonate (0.1 to 0.5%) and the solution stayed clear from one week to one month depending upon the concentration of sodium carbonate used in the vehicle. However, on standing at room temperature for a longer period of time, white crystalline material (free artelinic acid) crystallized out from all solutions with various concentrations of sodium carbonate. Heating of the solution at 40 ° C. accelerated the crystal formation. Thus, the crystal formation is temperature and sodium carbonate concentration dependent; higher temperature accelerates while higher sodium carbonate concentration delays the crystal formation.

The precipitates were identified by NMR and x-ray crystallography to be pure artelinic acid. No decomposition product was detected on standing at room temperature for three months.

Since sodium carbonate is a strong base, the use of higher concentration of sodium carbonate for intravenous formulation is not an option because of possible clinical complication associated with the use of high pH intravenous formulation and the concern of AL stability under high pH condition.

The inventors have sought to develop a new intravenous drug as replacement of quinidine which is an old and the only parenteral antimalarial available in the US.

The present invention is directed to artelinic acid/L-lysine salt. The artelinic acid/L-lysine salt of the invention has been under development by Walter Reed Army Institute of Research to treat severe and complicated malaria. It is very soluble in 0.1-0.5% L-lysine/saline solution (>160 mg/ml), more stable than artesunic acid in 5% NaHCO₃ aqueous solution, well tolerated by rats up to 160 mg/ml by intravenous injection and rapidly cleared the parasitemia in a rat model in three day treatment regimen at 20 to 80 mg/kg.

The present invention is directed to an intravenous (IV) formulation that solves the problems of the prior art because it ensures 100% bioavailability with immediate peak concentrations. AL is the only member of the artemisinin class with both good solubility and stability in water to be good candidate for development as intravenous antimalarial drug.

The US government and US Army, in particular, is in great need of new parenteral antimalarial drugs to effectively reduce the morbidity and mortality rate of soldiers with cerebral malaria, when deployed in the epidemic region of the world. Quinine intravenous injection is an old and the only parenteral antimalaria available in U.S.

Therefore, it is an object of the present invention to provide effective antimalarial drugs that overcome the drawbacks of previous antimalarial drugs.

It is another object of the present invention to present an artemisinin derivative that is stable and non-toxic.

It is another object of the present invention to present an artemisinin derivative that is suitable for intravenous injection.

These and other objects of the present invention shall become apparent upon further reading of the disclosure.

### SUMMARY OF THE INVENTION

The present invention is directed to an intravenous formulation of artelinic acid for treatment of severe and complicated malaria. The formulation is an artelinic acid/L-lysine salt that is soluble in L-lysine/saline solution. The formulation is stable in solution and non-toxic at recommended clinical dosages.

### DETAILED DESCRIPTION

Alternative intravenous formulation for AL was sought after the problems associated with the use of sodium artelinate in diluted sodium carbonate solution were uncovered.

Feasibility of using preformed AL salts with three basic amino acids, L-lysine, L-arginine or L-histidine for AL intravenous formulation were studied. The basicity of histidine is too weak to form water soluble AL-histidine salt. The arginine is a strong basic amino acid that forms highly water soluble AL-arginine salt. However, the arginine-AL aqueous solution turned to deep red color on standing at room temperature within two days to one week. Therefore, L-lysine was deemed the most feasible.

The L-lysine forms water-soluble AL-Lysine salt with AL. The solubility of AL-Lysine salt (AL/LY) in 0.3% L-lysine solution is >160 mg/ml. The bulk AL/LY salt and its aqueous solution are very stable. Nevertheless, the aqueous solution of AL/LY in normal saline alone formed precipitation and in 0.3% L-lysine solution developed a light yellow color on standing at room temperature for one week or longer. The concentration of artelinic acid in AL/LY formulation, however, does not change even after color change was observed.

Although a ready-to-use intravenous solution in a multiple dose vial is a convenient and preferable formulation, current formulation of dissolving preformed AL/LY salt in 0.3% of L-lysine/saline solution prior to administration has been used satisfactorily to assess the efficacy and toxicity of AL in rat, Aotus and Rhesus monkey models. AL/LY salt showed no toxicity at 80 mg/kg and was well tolerated at 160 mg/kg in rat by intravenous. No adverse effects on rat and dog veins were observed in a dose of up to 160 mg/kg in rat once a day for 3 days and at a dose of 12 mg/kg in dog once a day for 7 days.

### INJECTABLE SOLUTION

AL/LY is soluble in water, saline, glucose solution or any IV injectable (neutral or basic) water solution. However, an initially clear AL/LY water solution tends to turn into an un-injectable turbid solution on standing, due to weak acidity of pure water which converts small percent of AL/LY salt into a water insoluble artelinic acid. Therefore, for practical purpose, the preferred vehicle for intravenous injection is a normal saline or 5% glucose solution containing 0.1-0.5% L-lysine. The final osmolarity is around 290 mosm/kg.

Preliminary results in rat efficacy study indicated that AL/LY, at 20 mg/kg/day, nearly cleared the parasitemia at the end of three day treatment regimen. However, recrudescence took place in 3 days after treatment. At the higher dose, 40 mg/kg and 80 mg/kg, a complete parasitemia clearance was observed within 3 days of treatment and the parasite clearance lasted for over 7 days, when gradual increase in parasitemia resumed. The results confirmed the earlier reports that artemisinin derivatives as a group are quick acting antimalarial drugs and are potentially valuable drugs to reduce the mortality of patient with cerebral malaria.

The AL/YL in 0.3% L-lysine solution is much better tolerated in rat than the formulation using 5% sodium carbonate (formation of sodium artelinate in situ) as vehicle. The LD₅₀ of AL in 5% sodium carbonate is 67 mg/kg in male rat and 50 mg/kg in female rat. The preliminary results indicated that the AL/LY formulation is well tolerated by the rat at 160 mg/kg with no death recorded.

We found that the quality of commercially available L-lysine varies from company to company and from one lot to the other. The pure L-lysine is a white powder, but older lots of commercial L-lysine turn into a light yellow powder. The yellow impurity is poorly soluble in methanol and has different NMR spectrum from that of pure L-lysine. Therefore, it is necessary to pre-purify the commercial L-lysine before use.

The differences in price between commercially available (Aldrich, Milwaukee, Wis.) L-lysine ($160/100 gram) and L-lysine HCl salt ($32/500 grams) are quite substantial. The difficulties in the preparation, storage and long term stability of L-lysine are reasons behind the high cost of free L-lysine and exactly the same reasons which lead us to choose preformed AL/LY salt over free artelinic acid as the key ingredient in our preferred parenteral AL formulations.

AL/LY salt can be prepared readily from commercial grade L-lysine without having to use expensive GLP grade L-lysine with clinically acceptable purity. Furthermore, AL/LY salt is more readily soluble in saline solution than to dissolve AL in diluted L-lysine solution. The salt formation of AL with L-lysine is, in a sense, an indirect process of L-lysine purification which is much easier than purification of a basic amino acid such as L-lysine. An obvious alternative formulation to AL/LY salt is to dissolve AL directly in solution containing excess (1.1 equivalent mole or more) of L-lysine with clinically acceptable purity. However, the L-lysine with required purity is more expensive to prepare and more difficult to store than the AL/LY salt due to instability of L-lysine to air oxidation and carbonate formation during the process of purification and storage.

### MATERIALS AND METHODS

Partial Purification of L-lysine:

L-Lysine (Aldrich, Milwaukee, Wis.) 20 grams was suspended in 200 ml of absolute methanol. The suspension was filtered with a Whiteman No. 1 filter paper. The clear filtrate was evaporated to dryness under reduced pressure to give a white powder. The NMR (Bruker 600 MHz) showed the sample to be pure L-lysine. The freshly purified L-lysine was used to prepare the vehicle solution.

Preparation of Artelinic Acid/L-Lysine Salt (AL/LY Salt):

L-lysine (9 grams, Aldrich, Milwaukee, Wis.) was suspended in 100 ml of absolute methanol and filtered with a Whiteman No. 1 filter paper. The filtrate was added with stirring in small portions to an AL ethanol solution (25 grams of artelinic acid in 350 ml of absolute ethanol). The stirring was continued for an hour after the addition of L-lysine solution was completed. The white precipitates were collected, washed with 50 ml of fresh absolute ethanol and dried to give 30 grams of the desired AL/LY salt as white powder. Proton NMR spectra and elemental analyses results indicated the salt to be pure AL/LY salt containing equal mole (1:1) of AL and L-lysine and a half mole of water. No impurity signal was observed.

Anal: C₂₉H₄₄N₂O₉.1/2.H₂O=573, Calcd: C, 60.73; H, 7.85; N, 4.89. Found: C, 60.53., H, 7.80., N, 5.01.

The pH of a 50 mg/ml aqueous solution is close to pH 7.

Solubility in 0.3% L-lysine solution >160 mg/ml.

The chemical structure for artelinic acid/L-lysine salt is as follows:

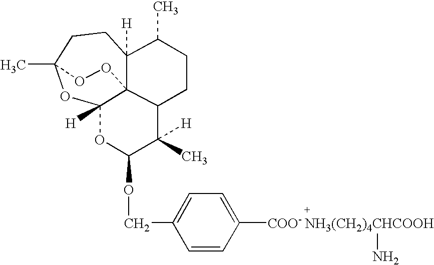

Formulation of Artelinic Acid/L-lysine Intravenous Injection:

1. The artelinic acid content of the pre-formulated artelinic acid/lysine salt (AL/LY) is equivalent to one gram of artelinic acid per 1.35 gram of AL/LY salt weight.

2. Vehicle solution for intravenous injection is prepared by dissolving 300 mg of the purified L-lysine in 100 ml of normal saline (3 mg/ml, 0.3%).

3. A 20 ml intravenous stock solution containing 40 mg/ml of AL/LY salt is prepared by dissolving 800 mg of the AL/LY salt in 20 ml of vehicle solution. The solution is sterilized by filtering through a syringe micro filter (0.2 mm, cellulose acetate) prior to use.

4. Intravenous solutions containing lower concentrations of AL can be made by dilution of aliquot stock solution (40 mg/ml) with the vehicle solution, followed by filtration through a syringe microfilter (0.2 mm, cellulose acetate) prior to use.

Alternate Intravenous Formulations:

1. The same procedure can be used to prepare intravenous solution for a concentration higher than 120 mg/ml in 0.3% L-lysine/saline solution. Half normal saline solution can be used to adjust the osmolarity of the intravenous solution to that of normal saline (˜290 mosm/kg).

2. Alternative formulation to this invention can be prepared by dissolving free artelinic acid in slightly higher than one equivalent mole of L-lysine/saline solution (0.1-0.5% excess). However, long term stability of pure L-lysine with clinically acceptable purity and its high cost are major problems that need to be overcome.

3. Further modification to the formulation can be made by dissolving AL/LY salt in solution containing 0.1-0.5% equivalent mole of purified L-lysine, followed by lyophylization to yield white powder. The product can be dissolved in normal saline, 5% glucose or half saline prior to use.

It should be noted that the quantities of the ingredients in the partial purification of L-lysine, the preparation of Artelinic acid/L-lysine salt, the formulation of Artelinic Acid/L-lysine intravenous injection or alternative formulations may be varied slightly or adjusted without taking away from the essence of the invention.

### OSMOLARITY OF AL/LY SALT SOLUTION

| | |
|---|---|
| 1. O.9% NaCl | 283 mosm |
| 2. 09% NaCl + 0.1% Lysine | 290 mosm |
| 3. 80 mg/ml AL/LY in 0.45% NaCl + 0.3% L-.Lysine | 302 mosm |
| 4. 40 mg/ml AL/LY in 0.45% NaCl + 0.3% L-lysine | 246 mosm |
| 5. 20 mg/ml AL/LY in 0.9% NaCl + 0.1% L-lysine | 308 mosm |
| 6. 160 mg/ml AL/LY in 0.3% L-lysine | 176 mosm |

The AL/LY salt formulation was used in rat with satisfactory results and has been tested in Aotus and Rhesus monkey models for assessment of efficacy and toxicity. The Severe Malaria STO and its consultants feel that rats and Rhesus monkeys are the best species for efficacy and toxicology testing of artemisinin compounds because the simian central nervous system is organized most similarly to that of humans.

The following protocol was taken from “Protocol Title: Toxicology of Single and Subacute Dosing of Candidate Intravenous Artemisinins in Rhesus Monkeys.

Protocols for Rhesus monkey studies are as follows:

Genes and Species: Macaca mulatta

Age: 4-10 years

Weight: 6-13 kg

Formulations:

1). Artelinate/lysine formulation—The artelinic acid content of the pre-formulated artelinic acid/L-lysine salt (powder) was equivalent to one gram of artelinate per 1.35 gram of salt weight. Vehicle solution for iv injection was prepared by dissolving 300 mg of purified L-lysine in normal saline (3 mg/mL or 0.3%).

A 20 mL iv stock solution containing 40 mg/mL of artelinic acid/L-lysine salt was prepared by dissolving 800 mg of AL/L-lysine salt in 20 mL of vehicle solution. After mixing, the solution was filter-sterilized by passage through a syringe filter (0.2 μm, cellulose acetate) prior to use. The artelinate/L-lysine solutions was stored refrigerated at approximately 5° C.

Additional iv solutions containing the required concentrations of AL were made by appropriate dilution of the 40 mg/ml stock solution with vehicle solution, followed by filtration through a syringe filter (0.2 mm, cellulose acetate) prior to use.

2). Vehicle Control (AL)—Vehicle control solution was prepared by dissolving 200 mg of L-lysine HCl in 20 mL of normal saline (0.9% NaCI) and sterilized by filter through syringe microfilter (0.2 mm, cellulose acetate) prior to use. The same volume of the control vehicle as that of the treated animals receiving the highest dosage was injected. The unused solution was kept in the refrigerator.

Route of Administration: Artelinate was administered iv because this is the anticipated route of administration of this compound to humans.

Each rhesus was given a single iv injection of the designated dose of AL/LY via a peripheral vein, administered as a slow iv push (3-4 ml/minute) into an iv of D₅W (5% dextrose in water). The dose volume administered was 4 ml/kg, which is well within safety limits for administration of an intravenous bolus to rhesus monkeys.

AL/LY Salt Subacute Toxicity Study:

AL/LY Salt Subacute Toxicity Study:

| Drug | Observation | | | | | |
|---|---|---|---|---|---|---|
| Dose | to Date | Neurologic | Activity | GI | Appetite | Weight Loss |
| Lysine | Day 22; | None | Normal | Normal | Normal | None (defined |
| Control | Complete | | | | | as <5%) |
| 5.9 | Day 22 | None | Normal | Normal | Normal | None |
| mg/kg | Complete | | | | | |
| 11.8 | Day 22; | None; | Normal | Normal; | Poor | Weight loss |
| mg/kg | complete | No tachycardia or | | Soft stool in | appetite in | >5% |
| | | bradycardia | | 50% on day | 50% | in 2 of 4 |
| | | | | 7-9 | | (13%, 5.2%) |
| 47.2 | Day 22; | 1. Somnolence, | Moderate | Vomiting | Poor | Weight loss > |
| mg/kg | complete | moderate noted | decreased | after dose in | appetite in | 5% in 3 of 4 |
| | | in all (transient | motor | 25%; | 75%, | animals |
| | | and non- | activity | No diarrhea; | improving | (8.2%, 7.6% |
| | | progressive) | lasting 1-3 | | after day | and 10.8%) |
| | | 2. Ataxia, transient | hours; | | 12; | |
| | | in 25%, post drug | noted | | Complete | |
| | | 3. Salivation in | during | | anorexia | |
| | | 25%, post drug | days 1-7 | | of 24 hrs | |
| | | | | | in 75% | |

| Drug | | | |
|---|---|---|---|
| Dose | Hematologic | Biochem | Urinalysis |
| Lysine | No toxicity | No toxicity | N/A |
| Control | No RBC loss or WBC changes | | |
| 5.9 | 1. No leukopenenia or | 1. Total protein | 1. Transient subclinical |
| mg/kg | neutropenia | drop of 7-15%. | hemoglobinuria (d1-d8); no red |
| | 2. RBC loss of 7-20%, none to | 25% below normal | urine |
| | anemia (<4.2 mil/μl) | limits. | |
| | 3. Slight reticulocytosis on | 2. Hypernatremia | |
| | day 10 in 100% | on day 4 in 50% | |
| 11.8 | 1. Relative neutropenia and | 1. Total protein drop | 1. Transient subclinical |
| mg/kg | leukopenia (25%) | (12-28%) | hemoglobinuria (d4-d8); no red |
| | 2. No leukocytosis/neutrophilia | 2. Alk Phos rise of | urine |
| | 3. 10-14% RBC loss | 20% in 3 of 4 | 2. No proteinuria |
| | 4. Slight Reticulocytopenia | 2. Trend to elevated | 3. Ketonuria in animal with |
| | | CPK on day 8 | marked anorexia |
| | | 3. Otherwise normal | |
| 47.2 | 1. Neutrophilia at 24 h (25%); no | 1. Alk phos rise > | 1. Red urine in all by 48 hours |
| mg/kg | neutropenia | 50% in 3 of 4 | 2. Clinical or subclinical |
| | 2. RBC loss of 15-21% at day 8 | 2. BUN rise | hemoglobinuria throughout in all |
| | (2 to anemia); progression to | 50% in 3 of | 2. 1-3+ proteinuria throughout |
| | day 15, then improvement | 4 on day 4 | Ketonuria in 75% |
| | 3. Reticulocytopenia with | 3. Creat rise 25% in | Glucosuria in 75% |
| | rebound on day 12-15 | 3 of 4 on day 4 | |
| | | 4. Total protein | |
| | | drop of 9-19%, | |
| | | nadir day 8 | |
| | | 5. Hypokalemia in | |
| | | 75% on day 8 | |

Summary of Rhesus Efficacy Trials:

1. AL/LY Efficacy in Uncomplicated Malaria (done with PK trial)

i. 10 splenectomized rhesus with mild P. coatneyi malaria (mean parasitemia 773K/μl or 11.8%) and one quinine control (773K/μl or 13.5%). All asymptomatic.

ii. 11.8 mg/kg loading dose given IV push over 3 minutes, then 5.9 mg/kg for 2 days

iii. 3 severe adverse events resulting in 2 deaths (DA467, DA660) from shock-like syndrome; no etiology determined; Gross pathology unremarkable; tissue histopathology consistent with severe malaria. Cause of death not obvious. Both had centrilobular liver degeneration with vacuolar changes and intracellular hyaline bodies of unclear etiology; forwarded to AFIP.

iv. Of 8 remaining monkeys, 25% (2 of 8) cleared parasitemia on two successive smears; all achieved at least 99% clearance. PCT 99 was 50.1 hours (range 33-72). Time to recrudescence was 24 and 36 hours, respectively.

v. Control animal received 7 days of IM quinine BID without incident; cleared parasitemia on day 8 and was cured.

2. Efficacy in Severe Malaria

a. Model limitations: very rapid escalation of parasitemia (to 35-55%) and deterioration in clinical signs. Therefore, a very narrow window for therapeutic intervention.

b. Quinine IM (BID for 7 days) controls: 80% (4 of 5) survived and severe malaria symptoms resolved in 3 days; parasitemia cleared in a mean of 5.6 days; all cured. If therapy delayed 24 hours, 50% died and the other had very slow recovery.

c. AL/LY: 6 doses checked in dose ranging (n=3 monkeys/group):

| Dose of AL (mg/kg) | Mortality | Parasite Clearance | Comments |
|---|---|---|---|
| 1.5 mg/kg load then 0.75 mg/kg/d × 2 | 67% | None; all >99% | Death <24 htrs after rx; No toxicities noted; |
| | (DA485) | | Necropsy c/w severe malaria and hemolysis; |
| | (DA656) | | hepatic centrilobular/midzonal necrosis c/w |
| | | | hypoxemia |
| 5.9 mg/kg load then 2.95 mg/kg/d × 2 | 33% | None; all >99% | No toxicities noted; Late death (day 15) |
| | (DA646) | | from chronic renal failure, purulent |
| | | | necrotizing myositis after IM quinine, and |
| | | | acute necrotizing enterocolitis |
| 8.8 mg/kg load then 4.4 mg/kg/d × 2 | 33% | None; all >99% | Death >24 hours; 2 doses AL given; 31% |
| | (DA664) | | drop in parasitemia; necropsy pending |
| 11.8 mg/kg load then 5.9 mg/kg/d × 2 | 33% | None; all >99% | Death <24 hours c/w malaria; necropsy c/w |
| | (DA555) | | severe malaria with acute ATN and |
| | | | intraluminal hemoglobin; No liver necrosis. |
| 23.6 mg/kg load then 11.8 mg/kg/d × 2 | 100% | N/A | Deaths at 18 hrs; 36 hrs and 114 hours; Late |
| | (DA532) | | death 48 hours after parasite clearance with |
| | (DA599) | | renal failure and jaundice; |
| | (DA684) | | Necropsies pending |
| 47.2 mg/kg load then 23.6 mg/kg/d × 2 | 67% | Parasitemia cleared | Deaths at 100 and 108 hours; both had |
| | (DA451) | for 11 days | cleared parasitemia; ˜48 hours; |
| | (DA649) | | Necropsy DA 451: few intraluminal |
| | | | protozoal parasites c/w resolving malaria. |
| | | | Kidneys: Severe ATN with massive |
| | | | intraluminal hemoglobin-like casts. Liver: |
| | | | Severe centrilobular bridging to submassive |
| | | | necrosis.GI: acute mild congestion. |
| | | | Other is pending |

Overall 56% mortality regardless of dose administered. If include the low parasitemia treatment group, 43% mortality.

The proposed dosage for humans is about 2-4 mg/kg/day given three times per day until symptoms are gone.

The method of administration for the composition is oral, intravenous, and intramuscular injection.

The vehicles that are acceptable as carriers for the composition are water, normal saline, various concentrations of saline or glucose solutions, and neutral or basic IV injectable vehicles.

Acceptable additives for the formulation are L-lysine, sodium chloride, glucose or injectable nutrients.

Quinine can also be co-administered in conjunction with said Artelinic Acid/L-lysine intravenous antimalarial formulation for the treatment of severe complicated malaria.

Having now fully described the invention, it will be apparent to one of ordinary skill in the art that many changes and modifications can be made thereto without departing from the spirit or scope of the invention as set for the herein.

### REFERENCES

(1). Paper 1: Lin, A. J.; Klayman, D. L.; Milhous, W. K. Antimalarial Activity of New Water-Soluble Dihydroartemisinin Derivatives. J. Med. Chem. 1987, 30, 2147-2150.

(2). A. J. Lin, D. L. Klayman, and W. K. Milhous, Novel Water-Soluble Antimalarial Dihydroartemisinin Derivatives, U.S. Pat. No. 4,791,135, Dec. 13, 1988.

(3). (a) China Cooperative Research Group on Qinghaosu and Its Derivatives as Antimalarials. The Chemistry and Synthesis of Qinghaosu Derivatives, Tradit Chin. Med 1982, 2, 9-16; (b) China Cooperative Research Group on Qinghaosu and Its Derivatives as Antimalarials. Clinical Studies on the Treatment of Malaria with Qinghaosu and its Derivatives. Tradit. Chin. Med. 1982, 2, 45-50.

(4). Ziffer, H.; Highet, R. J.; and Klayman, D. L. Artemisinin: An Endoperoxidic Antimalarial fro Artemisia annual L., Progress in the Chemistry of Organic Natural Products, 1997, 72, 121-214, Springer Wien, New York.

(5). (a) Klayman, D. L. Qinghaosu (Artemisinin): An Antimalarial Drug from China. Science, 1985, 228, 1049-1055; (b) Cuming, J. N.; Ploypradith, P.; Posner, G. H. Antimalarial Activity of Artemisinin (Qinghaosu) and Related Trioxanes: Mechanism(s) of Action. Advances Pharmacol. 1996, 37, 253-297; (c) Meshnick, S. R.; Taylor, T. E.; Kamchonwongpaisan, S. Artemisinin and the Antimalarial Endoperoxides: from Herbal Remedy to Targeted Chemotherapy. Microbiol. Revs. 1996, 60, 301-315.

(6). (a). Batty, K. T.; Ilett, K. F.; Davis, T. M. Chemical Stability of Artesunate Injection and Proposal for Its Administration by Intravenous Infusion. J. Pharm. Pharmacol. 1996, 48, 22-26., (b) Hien, T. T.; White, N. J. Quinghaosu, The Lancet, 1993, 341, 603-608.

(7). China Cooperative Research Group on Qinghaosu and Its Derivatives as Antimalarials. Metabolism and Pharmacokinetics of Qinghaosu and its Derivatives, J. Tradit. Chin. Med. 1982, 2, 25-30.

(8). Lee, I. S.; Hufford, C. D. Metabolism of Antimalarial Sesquiterpene Lactones. Pharmac. Ther. 1990, 48, 345-355.

(9). Karbwang, J., et al., Comparison of artemether and quinine in the treatment of severe falciparum malaria in south-east Thailand. Trans R Soc Trop Med Hyg, 1995. 89(6): p. 668-71.

(10). Murphy, S., et al., An open randomized trial of artemether versus quinine in the treatment of cerebral malaria in African children. Trans R Soc Trop Med Hyg, 1996. 90(3): p. 298-301.

(11). Taylor, T. E., et al., Rapid coma resolution with artemether in Malawian children with cerebral malaria. Lancet, 1993. 341(8846): p. 661-2.

(12). Taylor, T. E., et al., Intramuscular artemether vs intravenous quinine: an open, randomized trial in Malawian children with cerebral malaria. Trop Med Int Health, 1998. 3(1): p. 3-8.

(13). Walker, O., et al., An open randomized comparative study of intramuscular artemether and intravenous quinine in cerebral malaria in children. Trans R Soc Trop Med Hyg, 1993, 87(5): p. 564-6.

(14). Looareesuwan, S., et al., Efficacy and tolerability of a sequential, artesunate suppository plus mefloquine, treatment of severe falciparum malaria. Ann Trop Med Parasitol, 1995. 89(5): p. 469-75.

(15). Birku, Y., E. Makonnen, and A. Bjorkman, Comparison of rectal artemisinin with intravenous quinine in the treatment of severe malaria in Ethiopia. East Afr Med J, 1999. 76(3): p. 154-9.

(16). Lin, A. J.; Klayman, D. L.; Milhous, W. K. Antimalarial Activity of New Water-soluble Dihydroartemisinin Derivatives. J. Med. Chem. 1987, 30, 2147-2150.

(17). Nealon, C.; et al. Intramuscular Bioavailability and clinical Efficacy of Artesunate in Gabonese Children with severe Malaria, Antimicro. Agents and Chemother., 2002, 46, 3933- 3939.

(18). (a) Li, Q.; Peggins, J. O.; Fleckenstein, L. L.; Masonic, K.; Heiffer, M. H.; and Brewer, T. G. The Pharmacokinetics, and Bioavailability of Dihydroartemisinin, Arteether, Artesunic Acid and Artelinic Acid in Rats., J. Pharm. Pharmacol., 1998, 50, 173-182., (b) Leskovac, V.; Theoharides, A. D. Hepatic Metabolism of Artemisinin Drugs—1. Drug Metabolism in Rat Liver Microsomes, Comp. Biochem. Physiol. 1991, 99, 383-390.

(19). (a) Brewer, T. G.; Grate, S. J.; Peggins, J. O.; Weina, P. J.; Petfas, J. M.; Heiffer, M. H.; Schuster, B. G. Fatal Neurotoxicity of Arteether and Artemether. Am J. Trop. Med. Hyg. 1994, 51(3), 251-259; (b) Brewer, T. G.; Peggins, J. O.; Grate, S. J.; Petras, J. M.; Levine, B. S.; Weina, P. J. Neurotoxicity in Animals due to Arteether (AE) and Artemether (AM). Trans. R. Soc. Trop. Med. Hyg.; 1994, 88, Suppl 1:S33-6.

(20). Davis, et al., —Penetration of Dihydroartemisinin into Cerebrospinal Fluid after Administration of Intravenous Artesunate in Severe Falciparum Malaria, Antimicrobial Agents and Chemotherapy, 2003, 47, 368-370.

(21). Van Vianen, P. H.; Klayman, D. K.; Lin, A. J.; Lugt, C. B.; van Engen, A. L.; van der Kaay, H. J.; Mons, B. Plasmodium berghei: The Antimalarial Action of Artemisinin and Sodium Artelinate in vivo and in vitro, Studied by Flow Cytometry. Exp. Parasit. 1990, 70, 115-123.

(22). Tripathi, R.; Puri, S. K.; and Dutta, G. P.. Sodium β-Artelinate—A New Potential Gametocytocide. Exp. Parasit. 1996, 82, 251-254.

What is claimed is:

1. A composition comprising artelinic acid/L-lysine salt having the following formula:

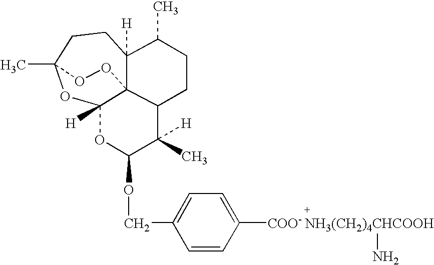

2. The composition of claim 1, wherein said artelinic acid/L-lysine salt is prepared by:
a) adding in small portions of filtered methanolic commercial grade L-lysine solution into an AL solution, said AL solution comprising artelinic acid and absolute ethanol;
b) forming a precipitate;
c) collecting and washing said precipitates with absolute ethanol; and
d) drying said precipitates to form artelinic acid/L-lysine salt.

3. An antimalarial formulation comprising: artelinic acid/L-lysine salt in solution.

4. The antimalarial formulation of claim 3, wherein said solution comprises a saline solution, a saline solution with 0.1- 0.5% L-lysine or glucose solution.

5. A method of forming an Artelinic Acid/L-lysine intravenous antimalarial formulation comprising:
a)preparing an artelinic acid/L-lysine salt;
b)preparing a vehicle solution, said vehicle solution comprising purified L-lysine in normal saline or 5% glucose; and
c)dissolving said artelinic acid/L-lysine salt in said vehicle solution to form said antimalarial formulation.

6. A method of forming an Artelinic Acid/L-lysine intravenous antimalarial formulation comprising:
a) purifying L-lysine;
b) preparing an artelinic acid/L-lysine salt;
c) preparing a vehicle solution, said vehicle solution comprising purified L-lysine in normal saline or 5% glucose;
d) dissolving said artelinic acid/L-lysine salt in said vehicle solution to form said antimalaria formulation.

7. The method of claim 6, wherein said L-lysine is purified by suspending L-lysine in absolute methanol to form a suspension; filtering said suspension to form a filtrate; and drying said filtrate to retrieve said L-lysine in a purified form.

8. The method of claim 6, wherein said artelinic acid/L-lysine salt is prepared by:
a) adding in small portions of filtered methanolic commercial grade L-lysine solution into an AL solution, said AL solution comprising artelinic acid and absolute ethanol;
b) forming a precipitate;
c) collecting and washing said precipitates with absolute ethanol; and
d) drying said precipitates to form artelinic acid/L-lysine salt.

9. The method of claim 6, wherein said vehicle solution is a 0.1-0.5% L-lysine solution.

10. A method of treating a patient with malaria comprising, administering to said patient the composition of claim 1.

11. The method of claim 10, wherein said composition is administered orally.

12. A method of treating a patient with malaria comprising, administering to said patient the formulation of claim 3.

13. The method of claim 12, wherein said formulation is administered intravenously, orally or intramuscularly.

14. The method of claim 12, wherein the formulation is given to said patient in a dose of 2-4 mg/kg/day given 3 times a day until symptoms disappear.

15. The method of claim 12, wherein quinine is administered in conjunction with said Artelinic Acid/L-lysine intravenous antimalarial formulation.

16. A powdered Artelinic Acid/L-lysine prepared by the process comprising:
a) dissolving AL/LY salt in solution containing 0.1-0.5% equivalent mole of purified L-lysine,
b) lyophylization said dissolved AL/LY salt solution to yield powder.

15. An antimalarial formulation comprising said lyophyilized powder of claim 14 dissolved in normal saline, 5% glucose, distilled water or half saline.
